(19) United States
(12) Patent Application Publication
(10) Pub. No.: US 2010/0092429 A1
(43) Pub. Date: Apr. 15, 2010

(54) Composition

(75) Inventors: Rautonen Nina, Espoo (FI); Arthur Ouwehand, Inkoo (FI); Julian Stowell, Kent (GB); Henna Makelainen, Yliskulma (FI)

Correspondence Address: STEPTOE & JOHNSON LLP, 1330 CONNECTICUT AVENUE, N.W., WASHINGTON, DC, 20036, US

(73) Assignee: Danisco A/S, Copenhagen K (DK)

(21) Appl. No.: 12/518,692

(22) PCT Filed: Dec. 10, 2007
(86) PCT No.: PCT/GB2007/004714

§ 371 Date: Dec. 7, 2009

(30) Foreign Application Priority Data
Dec. 11, 2006 (GB) 0624697.9
Dec. 11, 2006 (US) 60869368

(51) Int. Cl. A61K 35/74; A23C 9/123; A23C 9/13; A23C 9/152; A23L 1/29; A23L 1/30; A23L 2/39; A61P 1/00; A61P 37/00; A61P 17/00

(52) U.S. Cl. 424/93.4; 426/61

(57) ABSTRACT

The present invention provides a composition comprising: (a) one or more live Bifidobacterium lactis strains; and (b) a saccharide component; wherein the saccharide component comprises xylo-oligosaccharides with a degree of polymerisation of from 2 to 100.

The present invention relates to compositions and uses of such compositions.

Probiotics are dietary supplements containing live microbes, in particular bacteria, which potentially benefit a host by improving its intestinal microbial balance. A number of different microbes are used, the most common being lactic acid bacteria. Typically food compositions comprising such microbes are incorporated into fermented milk products such as yoghurts.

The rationale for probiotics and prebiotics is that a body contains an ecology of microbes, collectively known as the gut flora. Some circumstances (such as the use of antibiotics or other drugs, excess alcohol, stress, disease, or exposure to toxic substances) may alter the balance of the microbes. In such circumstances, the microbes that work well with the body may decrease in number, which may allows harmful competitors to thrive, to the detriment of the health of the body.

Probiotics are intended to assist the body's naturally occurring flora within the intestine. For example, they are sometimes recommended by doctors, and more frequently by nutritionists, after a course of antibiotics in order to assist the re-establishment of the body's natural flora.

Prebiotics were defined for the first time in 1995 as “non-digestible food-ingredients that beneficially affect the host by selectively stimulating the growth and/or activity of a limited number of bacteria in the colon” (Gibson, G. R., Roberfroid, M. B. J. Nutr. 125 (1995) 1401-1412). These substances are not digested or absorbed in the upper gastrointestinal tract, but they are fermented selectively in the colon. Prebiotics serve as targeted energy source for the beneficial microbes in the colon, principally lactobacilli and bifidobacteria, providing them a nutritional advantage in the very competitive colonic environment (Tuohy, K. M., et al. Curr. Pharmaceut. Design 11 (2005) 75-90).

Functional food can contain prebiotics, probiotics or a combination of these components. The fermentation of prebiotics by probiotic bacteria, mainly bifidobacteria and lactobacilli, is believed to benefit the health of the host. Prebiotics are believed to promote good health by having an effect on gut functionality, resistance to pathogen colonization, immunology, colon cancer, and lipid and mineral metabolism (Gibson, G. R., Roberfroid, M. B. J. Nutr. 125 (1995) 1401-1412).

U.S. Pat. No. 6,544,568 discloses a functional food comprising a baked part that comprises a prebiotic non-digestible fibre, and a non-baked part that comprises a probiotic live lyophilized lactic acid bacteria.

Crittenden, R. G., et al. Journal of Applied Microbiology 90 (2001) 268-278, disclosed the use of Bifidobacterium lactis Lafti™ B94 and resistant starch in a symbiotic yoghurt. In addition to resistant starch, this bacteria was also able to utilize a number of other prebiotic substances.

Despite these disclosures, there is a continuing need for more selective and efficient combinations of probiotics and prebiotics.

The present invention alleviates the problems of the prior art.

In one aspect the present invention provides a composition comprising:

(a) one or more live Bifidobacterium lactis strains; and

(b) a saccharide component;

wherein the saccharide component comprises xylo-oligosaccharides with a degree of polymerisation of from 2 to 100.

In a further aspect, the present invention provides a product for oral consumption comprising a composition of the present invention wherein the product is selected from dry oral supplements, liquid oral supplements, milk, yoghurt, cheese, ice-creams, products made of fermented cereals, infant formulae, powdered beverages, confectionary, nutrition bars and milk powders.

In a further aspect, the present invention provides a use of a composition according to the present invention, in the manufacture of a medicament to selectively increase the colonisation and/or the activity of Bifidobacterium lactis in the intestine of a subject.

In a further aspect, the present invention provides a means of selectively increasing the level of Bifidobacterium lactis during the fermentation of a probiotic containing food.

Xylo-Oligosaccharides

Xylo-oligosaccharides comprise molecules of a pentose sugar xylose which are connected by 1,4-β-linkages, but other linkages are also possible. The polymerisation degree of xylo-oligosaccharides refers to the number of xylose units. Thus, xylobiose consists of two molecules of xylose connected by 1,4-β-linkages and has a polymerisation degree of 2. Similarly, xylotriose has a polymerisation degree of 3. The degree of polymerization, or dp, is the number of repeat units in an average polymer chain at time t in a polymerisation reaction.

In a further aspect, preferably the saccharide component (b) comprises xylo-oligosaccharides with a polymerisation degree of 2 as its principal component. That is to say that the largest single group of xylo-oligosaccharides in such a saccharide component has a polymerisation degree of 2. Thus, for example, such xylo-oligosaccharides contain a higher proportion of xylo-oligosaccharides with a polymerisation degree of 2 than xylo-oligosaccharides with a polymerisation of 3. However, the proportion of xylo-oligosaccharides with a polymerisation degree of 2 in such xylo-oligosaccharides need not necessarily make up the majority of the xylo-oligosaccharides and may be less than 50%.

Preferably the proportion of xylo-oligosaccharides with a polymerisation degree of 2 in saccharide component (b) is at least 40%; preferably at least 45%; preferably at least 50%; preferably at least 55%: preferably at least 60%; preferably at least 65% preferably at least 70%; preferably at least 75% preferably at least 80%. These percentages are percentages by weight on a dried basis.

Preferably the xylo-oligosaccharides have a degree of polymerisation of 2.

In a further aspect, preferably the saccharide component (b) comprises xylo-oligosaccharides with a polymerisation degree of 3 as its principal component.

Preferably the proportion of xylo-oligosaccharides with a polymerisation degree of 3 in saccharide component (b) is at least 10%; preferably at least 12%; preferably at least 14%; preferably at least 15%; preferably at least 16%; preferably at least 17%; preferably at least 18%.

Preferably the proportion of xylo-oligosaccharides with a polymerisation degree of at least 4 in saccharide component (b) is at least 30%; preferably at least 35%; preferably at least 40%; preferably at least 45%; preferably at least 50%; preferably at least 55%; preferably at least 60%.

Preferably the proportion of xylo-oligosaccharides with a polymerisation degree of at least 5 in saccharide component (b) is less than 90%; preferably less than 85%; preferably less than 80%; preferably less than 75%; preferably less than 70%; preferably less than 65%; preferably less than 60%.

Preferably the proportion of xylo-oligosaccharides in saccharide component (b) with a polymerisation degree of from 2 to 10 is at least 60%; preferably at least 65%; preferably at least 70%; preferably at least 75%; preferably at least 80%; preferably at least 85%; preferably at least 90%; preferably at least 95%.

Preferably the proportion of xylo-oligosaccharides in saccharide component (b) with a polymerisation degree of 1 is less than 80%; preferably less than 70%; preferably less than 60%; preferably less than 50%; preferably less than 40%; preferably less than 35%; preferably less than 30%; preferably less than 25%; preferably less than 20%.

Preferably the proportion of xylo-oligosaccharides in saccharide component (b) with a polymerisation degree of from 1 to 2 is less than 80%; preferably less than 70%; preferably less than 60%; preferably less than 50%; preferably less than 40%; preferably less than 35%; preferably less than 30%; preferably less than 25%; preferably less than 20%.

Preferably the xylo-oligosaccharides have a degree of polymerisation of from 2 to 10.

In a further aspect, preferably the proportion of xylo-oligosaccharides with a polymerisation degree of at least 5 in saccharide component (b) is greater than 45%; preferably greater than 50%; preferably greater than 55%; preferably greater than 60%; preferably greater than 65%; preferably greater than 70%; preferably greater than 75%; preferably greater than 80%; preferably greater than 85%; preferably greater than 90%.

In one aspect, preferably the xylo-oligosaccharides is xylan.

Preferably the xylan has a degree of polymerisation of at least 30. Preferably the xylan is selected from a xylan with a degree of polymerisation of from 35 to 40; a xylan with a degree of polymerisation of 41 to 50; a xylan with a degree of polymerisation of from 51 to 60; a xylan with a degree of polymerisation of from 61 to 70; and a xylan with a degree of polymerisation of from 71 to 80.

Xylo-oligosaccharides include xylan which may be obtained from corn, sugar cane, bamboo, cottonseed and wood. Preferably, the xylo-oligosaccharides are obtained from wood. Xylo-oligosaccharides with lower degrees of polymerisation than xylan may be prepared by enzymatic hydrolysis of xylan. The enzymatic hydrolysis of xylan may be carried out using xylanase EC 3.2.1.8. Alternatively, chemical degradation of xylan may be preformed using steam, diluted solutions of mineral acids (e.g. phosphoric acid) or alkaline solutions. Such chemical and enzymatic steps may be used sequentially. Separation and purification of the xylo-oligosaccharides may be carried out by a variety of processes. These processes include vacuum evaporation to remove volatile impurities, such as acetic acid; and solvent extraction with organic solvents. Separation of xylo-oligosaccharides within a given dp range has been carried out with membranes and also, with ethanolic solutions of different concentration. Adsorption, ion-exchange and chromatographic separation techniques may also be used to purify the xylo-oligosaccharides. A variety of xylo-oligosaccharides are commercially available.

U.S. Pat. No. 6,942,754 discloses an enzymatic method of preparing xylo-oligosaccharides from lignocellulose pulp.

Bifidobacterium Lactis

Any Bifidobacterium lactis strain may be used.

Preferably the one or more live Bifidobacterium lactis strains are selected from, but not restricted to, B. lactis BI-04, B. lactis Bi-07, B. lactis 420, B. lactis DN 173 010, B. lactis HN019, B. lactis Bb-12, B. lactis DR10, B. lactis DSM10140, B. lactis LKM512, B. lactis DSM 20451 and mixtures thereof.

Preferably the one or more live Bifidobacterium lactis bacteria strains are selected from B. lactis BI-04, B. lactis Bi-07, B. lactis 420, B. lactis Bb-12, B. lactis DN 173 010, B. lactis HN019 and mixtures thereof.

Preferably when the composition is a food composition, the food composition comprises from 1×10⁶ to 1×10¹² Colony Forming Units (CFU) per serving of Bifidobacterium lactis strains. Preferably, the food composition comprises from 10⁷-10¹⁰ CFU per serving of Bifidobacterium lactis strains.

Product for Oral Consumption

In a further aspect, the invention relates to a product for oral consumption comprising a composition as described herein. Such products for oral consumption may include foodstuffs, or oral supplements. The composition described herein is a component of such a product for oral consumption.

Preferably the product for oral consumption is selected from dry oral supplements, liquid oral supplements, milk, yoghurt, cheese, ice-creams, products made of cereals, infant formulae, powdered beverages, confectionary, nutrition bars and milk powders. Preferably the dry oral supplement is a tablet or a pill. Preferably the cereal is muesli.

Preferably the product for oral consumption comprises from 2 to 10 g per serving, or per dose, of xylo-oligosaccharides; preferably from 3 to 9 g per serving, or per dose, of xylo-oligosaccharides; preferably from 4 to 8 g per serving, or per dose, of xylo-oligosaccharides; preferably from 5 to 7 g per serving, or per dose, of xylo-oligosaccharides. Preferably the above dose of xylo-oligosaccharides is a daily dose.

Preferably the product for oral consumption comprises 5 g per serving, or per dose, of xylo-oligosaccharides.

Preferably the product for oral consumption comprises from 1×10⁶ to 1×10¹² Colony Forming Units (CFU) per serving, or per dose, of Bifidobacterium lactis strains. It is believed that below this range the amount of Bifidobacterium lactis would not be efficient; and to use amounts of Bifidobacterium lactis above this range would require too large volume of product for oral consumption for a human. Preferably, the product for oral consumption comprises from 10⁷-10¹⁰ CFU per serving, or per dose, of Bifidobacterium lactis strains. Preferably the above dose is a daily dose.

Preferably the product for oral consumption is a yoghurt. Preferably the yoghurt comprises from 10⁶ to 10⁸ CFU/ml per serving of Bifidobacterium lactis strains.

The product for oral consumption may further comprise components selected from preservatives, stabilisers, dyes, antioxidants, suspending agents and flavouring agents. Examples of preservatives include sodium benzoate, sorbic acid and esters of p-hydroxybenzoic acid.

Pharmaceutical Composition

In one aspect, the composition described herein is a pharmaceutical composition, which further comprises a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Kit

In a further aspect, the present invention provides a kit comprising a first vessel comprising one or more live Bifidobacterium lactis strains; and a second vessel comprising a saccharide component wherein the saccharide component comprises xylo-oligosaccharides with a degree of polymerisation of from 2 to 100.

Hence, the components of the compositions described herein may be provided in the form of a kit. The components may be provided for simultaneous, sequential or separate administration. The first vessel of the kit may comprise one or more Bifidobacterium lactis strains additionally comprising any of the further features relating to the Bifidobacterium lactis strains that are described herein in relation to the composition. Similarly, the second vessel of the kit may comprise a saccharide component additionally comprising any of the further features relating to the saccharide component and the xylo-oligosaccharides that are described herein in relation to the composition.

Preferably, the kit comprises Bifidobacterium lactis strains that are incorporated into, but not limited to, a pill or into yoghurt.

Preferably, the kit comprises xylo-oligosaccharides that are incorporated into a foodstuff selected from, but not limited to, fruit juice and products made of cereals. Preferably the cereal is muesli.

Preferably, the kit also comprises instructions. These instructions may relate to the recommended mode or order of administration of the components of the kit.

Uses

In a further aspect, the present invention provides a use of a composition or a kit as described herein in the manufacture of a medicament to selectively increase the colonisation and/or the activity of Bifidobacterium lactis in the intestine of a subject.

The composition or kit as described herein, may be used to increase the levels of Bifidobacterium lactis in a fermented food.

The composition or kit as described herein, may be used in the manufacture of a medicament to reduce or inhibit the colonisation of Clostridium perfingens in the intestine of a subject.

The composition or kit as described herein, may be used in the manufacture of a medicament to reduce or inhibit the colonisation of Salmonella typhimurium in the intestine of a subject.

The composition or kit as described herein, may be used in the manufacture of a medicament to reduce or inhibit the colonisation of enteropathogenic Escherichia coli in the intestine of a subject.

The composition as described herein, may also be used in a product for oral consumption selected from dry oral supplements, liquid oral supplements, milk, yoghurt, cheese, ice-creams, products made of fermented cereals, infant formulae, powdered beverages, confectionary, nutrition bars and milk powders

The composition or kit as described herein, may be used in the manufacture of a medicament to reduce atopic eczema.

The composition or kit as described herein, may be used in the manufacture of a medicament for the treatment of diarrhea.

The composition or kit as described herein, may be used in the manufacture of a medicament to enhance immune function.

The composition or kit as described herein, may be used in the manufacture of a medicament to improve bowel function.

Method

A method for selectively increasing the colonisation of Bifidobacterium lactis in the intestine of a subject, by orally administering to the subject a composition or kit as described herein.

A method for reducing or inhibiting the colonisation of Clostridium perfingens in the intestine of a subject, by orally administering to the subject an effective amount of a composition or kit as described herein.

A method for reducing or inhibiting the colonisation of Salmonella typhimurium in the intestine of a subject, by orally administering to the subject an effective amount of a composition or kit as described herein.

A method for reducing or inhibiting the colonisation of Escherichia coli in the intestine of a subject, by orally administering to the subject an effective amount of a composition or kit as described herein.

A method for reducing atopic eczema in a subject by orally administering to the subject an effective amount of a composition or kit as described herein.

A method for treatment of diarrhea in a subject, by orally administering to the subject an effective amount of a composition or kit as described herein.

A method for enhancing immune function in a subject, by orally administering to the subject an effective amount of a composition or kit as described herein

A method for improving bowel function in a subject, by orally administering to the subject an effective amount of a composition or kit as described herein

Preferably the subject is an animal or a human.

Preferably the subject is a mammal, a fish or poultry.

Preferably the subject is a mammal, preferably a human.

Saccharide Component (b)

The saccharide component (b) may contain less than 6.5% by weight of monosaccharides; preferably less than 6.0% by weight of monosaccharides; preferably less than 5.5%; preferably less than 5.0%; preferably less than 4.5%; preferably less than 4.0%; preferably less than 3.5%; preferably less than 3.0%; preferably less than 2.5%; preferably less than 2.0%; preferably less than 1.5%; preferably less than 1.0%; preferably less than 0.5%.

In a further aspect, the saccharide component (b) contains from 0% to 6.9% by weight of monosaccharides. Preferably the saccharide component (b) contains from 0 to 5.0% by weight of monosaccharides; preferably from 0 to 4.0%; preferably from 0 to 3.0%; preferably from 0 to 2.0%; preferably from 0 to 1.0%.

In a further aspect, the saccharide component (b) contains substantially no monosaccharides.

The present invention will now be described in further detail by way of example only with reference to the accompanying figures in which:

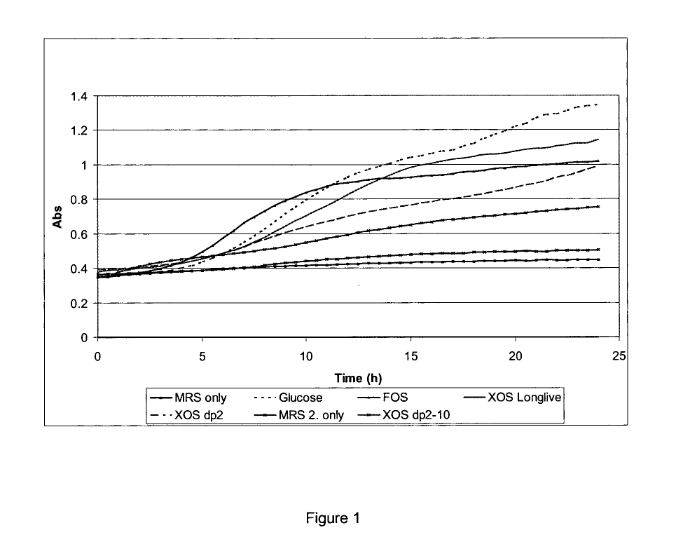

FIG. 1 shows growth curves for Bifidobacterium lactis BI-04 on a range of prebiotics;

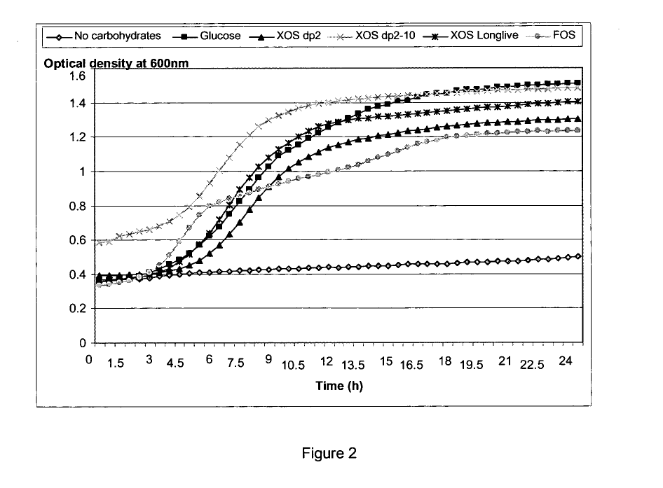

FIG. 2 shows further growth curves for Bifidobacterium lactis BI-04 on a range of prebiotics;

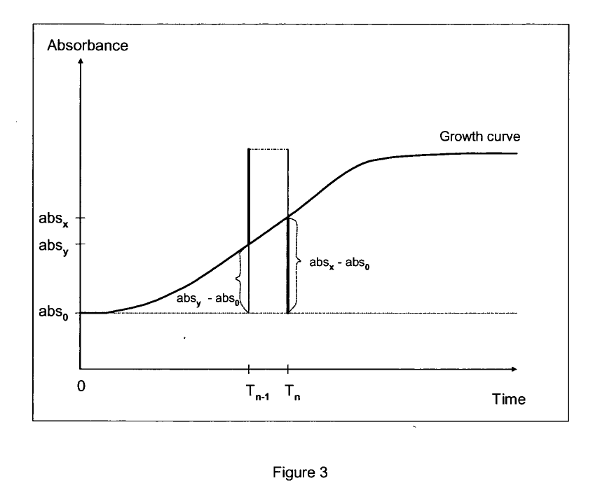

FIG. 3 shows an illustrating picture how to calculate the area under the growth curve;

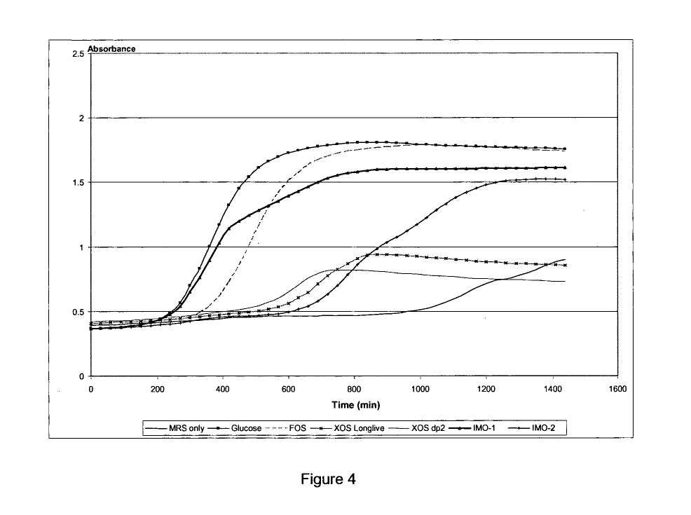

FIG. 4 shows growth curves for Lactobacillus acidophilus on a range of prebiotics;

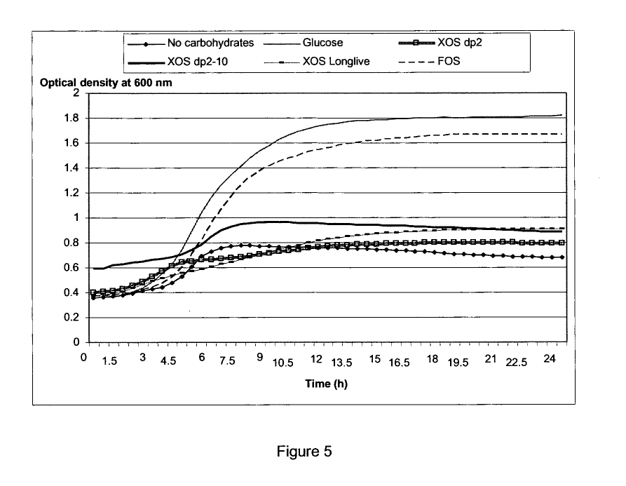

FIG. 5 shows further growth curves for Lactobacillus acidophilus on a range of prebiotics;

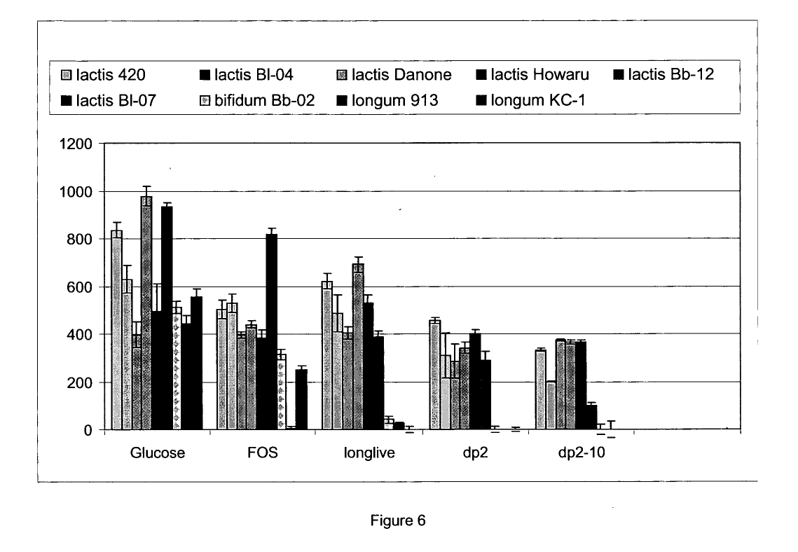

FIG. 6 shows a bar graph illustrating the rate of bacterial growth for various strains of Bifidobacteria on a range of single carbohydrates;

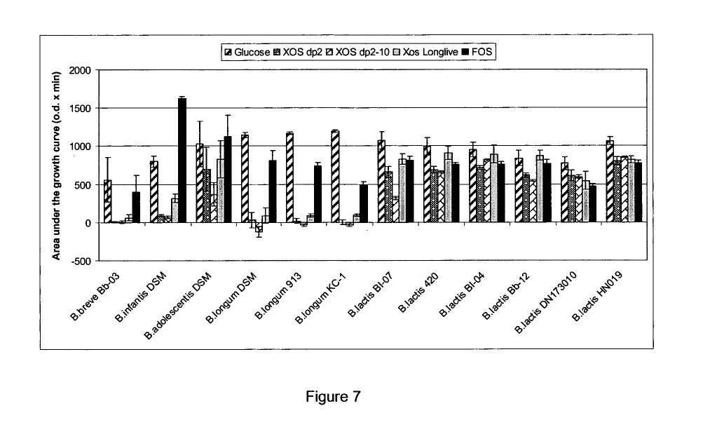

FIG. 7 shows a bar graph illustrating the rate of bacterial growth for various strains of Bifidobacteria on a range of single carbohydrates;

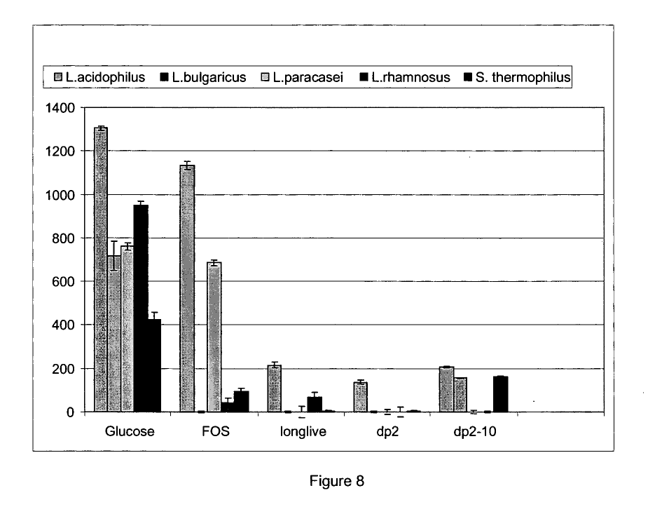

FIG. 8 shows a bar graph illustrating the rate of bacterial growth for various strains of bacteria on a range of single carbohydrates;

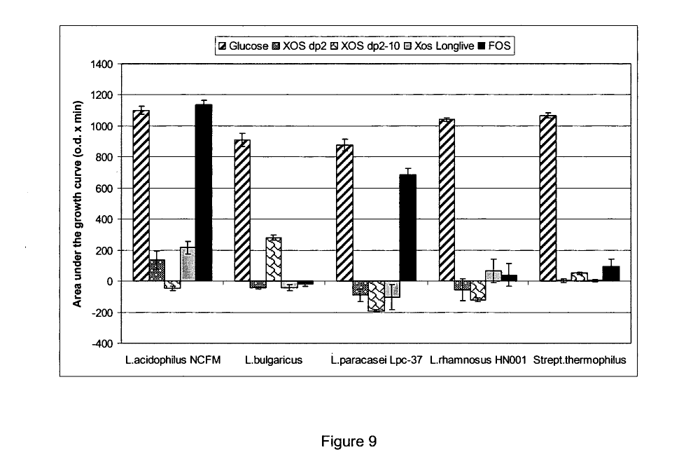

FIG. 9 shows a bar graph illustrating the rate of bacterial growth for various strains of bacteria on a range of single carbohydrates;

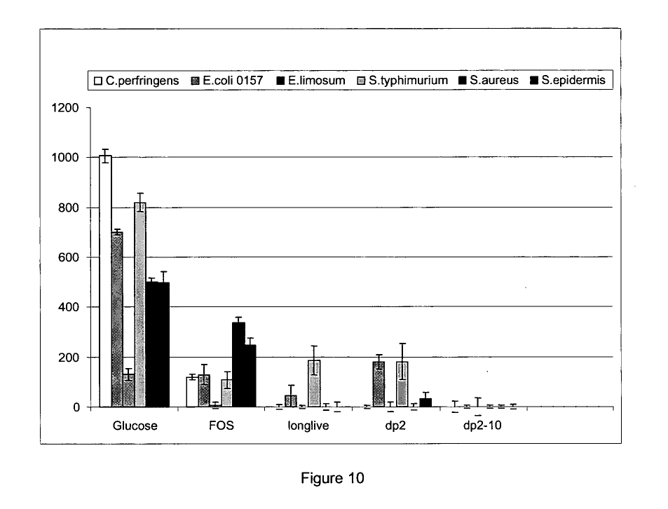

FIG. 10 shows a bar graph illustrating the rate of growth for various pathogens on a range of single carbohydrates;

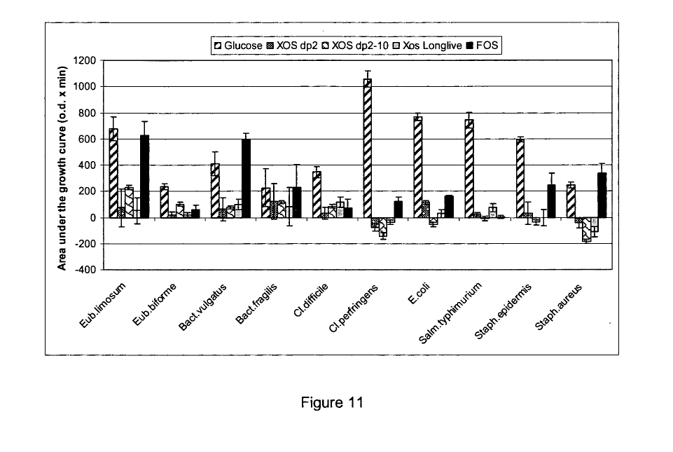

FIG. 11 shows a bar graph illustrating the rate of growth for various pathogens on a range of single carbohydrates;

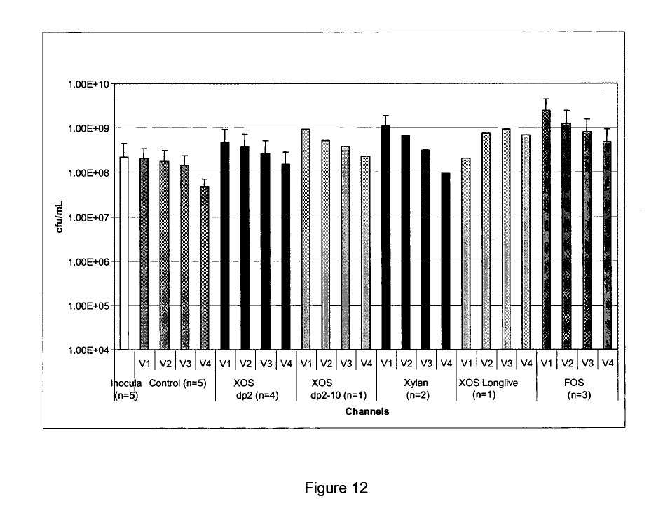

FIG. 12 shows a bar graph illustrating a colon simulation for Bifidobacteria with a range of single carbohydrates;

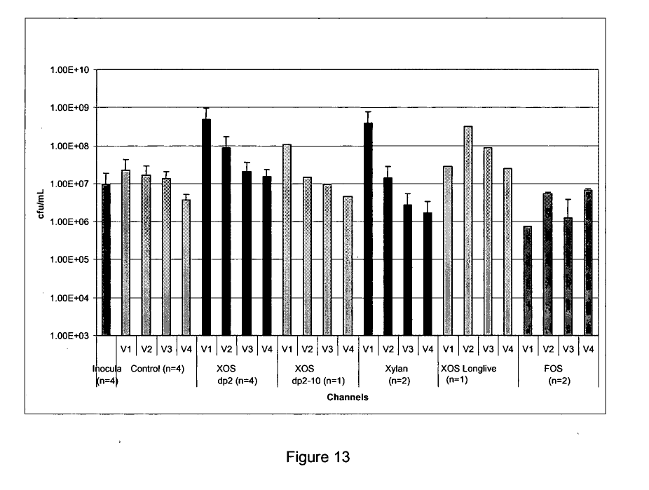

FIG. 13 shows a bar graph illustrating a colon simulation for Bifidobacterium lactis with a range of single carbohydrates;

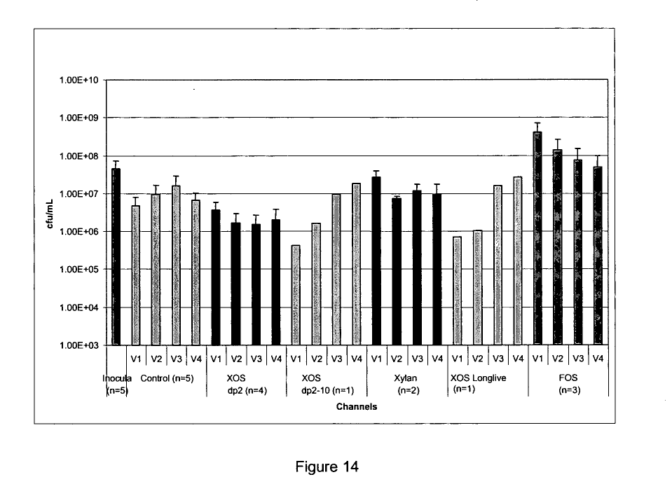

FIG. 14 shows a bar graph illustrating a colon simulation for Bifidobacterium longum with a range of single carbohydrates;

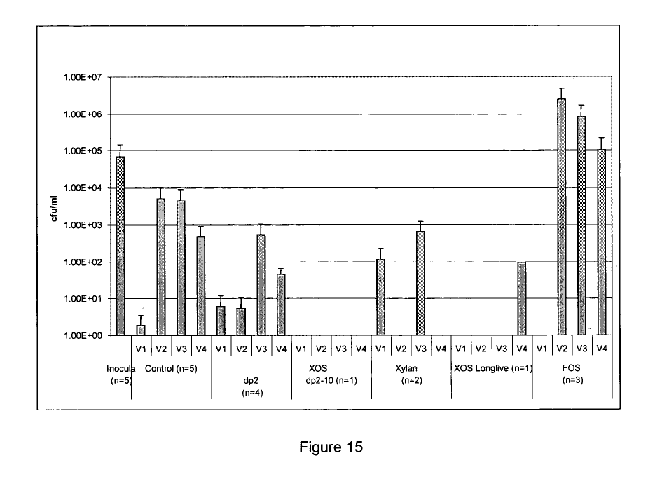

FIG. 15 shows a bar graph illustrating a colon simulation for Clostridium perfringens with a range of single carbohydrates; and

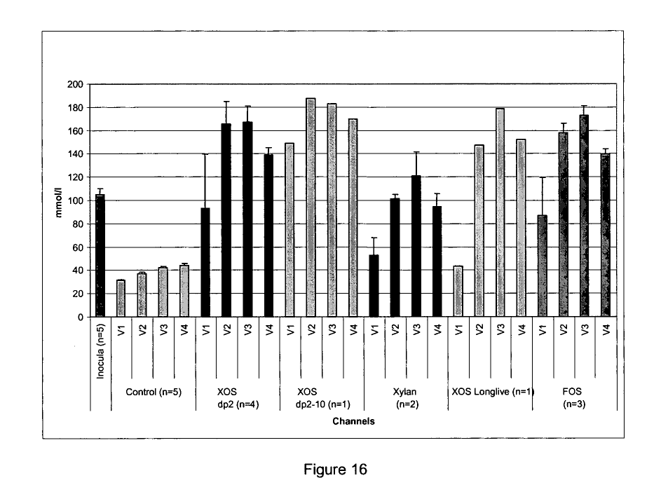

FIG. 16 shows a bar graph illustrating a colon simulation for total short chain fatty acids with a range of single carbohydrates.

The present invention will now be described in further detail in the following examples.

### EXAMPLES

Longlive 041021, 95P is available from Shandong Longlive, China.

RaftiloseP95 is available from Orafti, Belgium.

Alpha-D-Glucose is available from Serva, Germany.

de Man Rogosa Sharpe culture medium (MRS) is available from (LabM).

MRS 2 is MRS culture medium without glucose.

Tryptic Soy Broth culture medium (TSB) is available from Becton Dickinson, France.

Bifidomedium (Bif58), recipe is available from DSM, Deutsche Sammlung von Mikroorganismen.

Bifidobacterium lactis BI-07 is available from Danisco NS.

Bifidobacterium lactis BI-04 is available from Danisco NS.

Bifidobacterium lactis HN019 (Howaru) is available from Danisco A/S.

Bifidobacterium lactis DN 173 010 is available from Groupe Danone.

Bifidobacterium lactis Bb-12 is available from Christian Hansen A/S.

Bifidobacterium lactis 420 is available from Danisco A/S.

Bifidobacterium breve Bb-03 is available from Danisco A/S.

Bifidobacterium longum KC-1 is available from Danisco A/S.

Bifidobacterium longum 913 (Wisby) is available from Danisco A/S.

Lactobacillus acidophilus NCFM is available from Danisco A/S.

Lactobacillus bulgaricus 1260 is available from Danisco A/S.

Lactobacillus paracasei Lpc-37 is available from Danisco A/S.

Lactobacillus rhamnosus HN001 (Howaru) is available from Danisco A/S.

Streptococcus thermophilus 715 is available from Danisco A/S.

Other microbes used in the experiment are available from culture collections:

ATCC=American Type Culture Collection

DSM=Deutsche Sammlung von Mikroorganismen and Zellkulturen

CCUG=Culture Collection, University of Göteborg, Sweden

EELA=Finnish Food Safety Authority

The prebiotic candidates and their compositions are listed in Table 1.

Abbr.=Abbreviation, Ster.=Sterilization procedure, F=filtration, UV=ultraviolet radiation.

TABLE 1

| Group | Identifier | Abbr. used | Composition | Ster. |
|---|---|---|---|---|
| Xylo-oligosaccharide | Longlive | XOS | 84% XOS (43% dp2, 30% | F |
| (XOS) | 041021, 95P | Longlive | dp3, 10% dp4, 17% dp ≧ 5); | |
| | | | 13.5% other dp1 | |
| | dp 2 | XOS dp2 | 100% XOS (7% dp1, 82% | F |
| | | | dp2, 10% dp3, 1% dp4) | |
| | dp 2-10 | XOS dp2-10 | 99% XOS (13% dp2, 19% | UV |
| | | | dp3, 11% dp4, 60% dp ≧ 5) | |
| | Xylan | Xylan | 97% XOS dp ≧ 5 | UV |
| Isomalto-oligosaccharide | IMO-500 | IMO-1 | 51% IMO | F |
| (IMO) | | | | |
| | IMO-900 | IMO-2 | 45% IMO, 2% dp 1, 45% | F |
| | | | other non-fermentable dp > 2 | |
| Fructo-oligosaccharide | Raftilose P95 | FOS | 95.5% FOS dp2-6; 4.5% | F |
| | | | other dp2 | |
| Glucose | Alpha-D- | GLU | 100% Glucose | F |
| | Glucose | | | |

The prebiotics were dissolved in 10% concentrated stock solutions, sterilized either by filtration (0.2 μm Minisart NML, Sartorius AG, Germany) or by UV-radiation (30 s, 120 mJ/cm²) (XL-1500 UV Crosslinker, Spectronics Corporation, US) depending on the length of the carbohydrate chains and stored at +4° C. in aerobic conditions. The tested bacterial strains and their growth media used in the cultivation (the medium in parentheses is the medium used in the first pre-cultivation) are shown in Table 2. Bifidobacteria, lactobacilli, and Strep. thermophilus were grown in MRS medium and other bacteria in TSB.

All cultures were stored at −70° C. in bead vials (Technical Service Consultants, UK). For the subcultures, bacteria were inoculated from two beads to 5 ml of their appropriate growth medium (Table 2). The precultures were grown from frozen stocks for 24-48 h at 37° C. under anaerobic conditions in appropriate media, until they were dim. Then the bacteria were inoculated further to MRS or TSB (Table 2) and incubated for another 24 h at 37° C. These suspensions were used for growth intensity measurements

TABLE 2

| | | | | Growth | |
|---|---|---|---|---|---|
| Genera | Species | Strain | Origin | Medium | Remark |
| Bacteroides | fragilis | ATCC 25285 | Human | MRS | — |
| | vulgatus | DSM 1447 | intestinal | (Meat) | — |
| Bifidobacterium | adolescentis | DSM 20083 | bacteria | MRS | — |
| | breve | Bb-03 | | (Bif58) | Probiotic |
| | lactis | Bl-07 | | | |
| | | Bl-04 | | | |
| | | 420 | Unknown | | |
| | | DN 173 010 | Dairy | | |
| | | HN019 | | | |
| | | Bb-12 | Unknown | | |
| | longum | 913 | Human | | |
| | | KC-1 | intestinal | | |
| | | DSM 20019 | bacteria | | — |
| | infantis | DSM 20088 | | | — |
| Clostridium | perfringens | ATCC 13124 | | TSB | Pathogen |
| | difficile | ATCC 9689 | | | Pathogen |
| Escherichia | coli 0157:H2 | 0157:H2 | | | Pathogen |
| Eubacterium | limosum | ATCC 8486 | | MRS | — |
| | biforme | DSM 3989 | | (Meat) | — |
| Lactobacillus | acidophilus | NCFM 145 | | MRS | Probiotic |
| | paracasei | Lpc-37 | | | |
| | rhamnosus | HN001 | Dairy | | |
| | bulgaricus | 1260 | | | Yoghurt starter |
| Salmonella | typimurium | EELA 4185/96 | Chicken | TSB | Pathogen |
| Staphylococcus | aureus | ATCC 10990 | Human skin | | — |
| | epidermis | CCUG 37527 | bacteria | | — |
| Streptococcus | thermophilus | 715 | Dairy | MRS | Yoghurt starter |

In Vitro Growth of Probiotics on Prebiotics

General Method for Determining the Rate of Bacterial Growth on a Single Carbohydrate

Anaerobic growth was measured with an automatic on-line turbidometer (Bioscreen C, Labsystems, Finland), which records kinetic changes in the absorbance of the liquid samples in a multiwell plate. Each well of the plate was filled with 20 μl 10 w/v-% prebiotic solution in aerobic conditions, subsequently 180 μl 1 v/v-% early stationary phase test bacteria in its appropriate culture medium were added anaerobically. The control wells included only 200 μl 1 v/v-% early stationary phase test bacteria or the same medium used for culturing the bacteria in question. The final prebiotic concentration was 1 w/v-%.

All strains were incubated at 37° C. for 24 h and the absorbance (OD 600 nm) was measured every 30 min. Plates were shaken for 10 s before every measurement. Two different sets of experiments with five replicates were performed for each strain and carbohydrate combination.

The rate of bacterial growth on a single carbohydrate source was determined by calculating the area under the growth curve (24 h) from the absorbance results automatically processed by the software (BioLink, Version 5.07, Labsystems, UK). The similar analysis was performed by Jaskari et al. Appl. Microbiol. Biotechnol. 49 (1998) 175-181. The area under the growth curve was calculated for each well with the help of symmetric rectangles, which were drawn from absorbance values measured at a consecutive time points. This is illustrated in the FIG. 1.

The half of the area of this rectangle is the area under the growth curve between the consecutive time points and this can be written:

$$A = \frac{( {T}_{n} - {T}_{n - 1} ) \cdot ⌊ ( {abs}_{x} - {abs}_{0} ) + ( {abs}_{y} - {abs}_{0} ) ⌋}{2} \qquad ( 1 )$$

All the areas from the measurement period (24 h, measurement in every 30 min) were summed up to get the area under the growth curve for the each well's growth curves. The growth in the control medium (basal growth medium without added carbohydrates) was subtracted from results as baseline growth (abs₀). The variation between the parallel results of the each bacteria and prebiotic combination was calculated using the standard error of the mean (SE). The averages of the areas under the growth curve for ten parallel wells were used to calculate the SE as following:

$$SE = \frac{σ}{\sqrt{n}} , \qquad ( 2 )$$

where n=number of parallel subjects

$$σ = standard deviation = \sqrt{\frac{1}{n} {\sum }_{i = 1}^{n} {( {x}_{i} - \overline{x} )}^{2}} , \qquad ( 3 )$$

where n=number of parallel subjects
   xᵢ=the value of the subject i
   x̅=average value of parallel subjects.

Growth of Bifidobacterium lactis BI-04

The growth of Bifidobacterium lactis BI-04 on a range of probiotics was carried out using the general method. The results for these are shown in Table 3 and the growth curve obtained from this data is shown in FIG. 1.

TABLE 3

| | | | | | XOS | MRS 2. | XOS |
|---|---|---|---|---|---|---|---|
| Time (h) | MRS only | Glucose | FOS | XOS Longlive | dp2 | only | dp2-10 |
| 0 | 0.3501 | 0.345 | 0.3454 | 0.3778 | 0.3909 | 0.3622 | 1.1976 |
| 0.5 | 0.3557 | 0.3508 | 0.351 | 0.3854 | 0.3947 | 0.3652 | 1.1947 |
| 1 | 0.3601 | 0.3557 | 0.3571 | 0.3895 | 0.3972 | 0.3679 | 1.2135 |
| 1.5 | 0.3642 | 0.3612 | 0.3637 | 0.394 | 0.4012 | 0.3703 | 1.2288 |
| 2 | 0.3675 | 0.366 | 0.3724 | 0.3992 | 0.4056 | 0.3725 | 1.2467 |
| 2.5 | 0.3705 | 0.3717 | 0.3828 | 0.4041 | 0.4106 | 0.3749 | 1.2586 |
| 3 | 0.374 | 0.3794 | 0.3963 | 0.4109 | 0.4173 | 0.3773 | 1.2698 |
| 3.5 | 0.3773 | 0.3884 | 0.4128 | 0.4186 | 0.4236 | 0.3799 | 1.2781 |
| 4 | 0.3806 | 0.4001 | 0.4344 | 0.4276 | 0.4319 | 0.3822 | 1.2869 |
| 4.5 | 0.3845 | 0.4149 | 0.4606 | 0.4393 | 0.4417 | 0.3847 | 1.2947 |
| 5 | 0.38835 | 0.43625 | 0.4961 | 0.45375 | 0.45435 | 0.3876 | 1.30125 |
| 5.5 | 0.3922 | 0.4576 | 0.5316 | 0.4682 | 0.467 | 0.3905 | 1.3078 |
| 6 | 0.396 | 0.4843 | 0.575 | 0.4863 | 0.4836 | 0.3943 | 1.3143 |
| 6.5 | 0.399 | 0.5156 | 0.6169 | 0.5039 | 0.5022 | 0.3985 | 1.321 |
| 7 | 0.402 | 0.549 | 0.6626 | 0.5254 | 0.5227 | 0.403 | 1.3262 |
| 7.5 | 0.404 | 0.5855 | 0.6971 | 0.5512 | 0.5442 | 0.4088 | 1.3347 |
| 8 | 0.4065 | 0.6269 | 0.7295 | 0.5788 | 0.5656 | 0.4159 | 1.3427 |
| 8.5 | 0.4091 | 0.6688 | 0.7652 | 0.6132 | 0.5867 | 0.4225 | 1.3518 |
| 9 | 0.4109 | 0.7126 | 0.7909 | 0.6414 | 0.6052 | 0.4296 | 1.361 |
| 9.5 | 0.413 | 0.7528 | 0.816 | 0.6754 | 0.6228 | 0.4356 | 1.3715 |
| 10 | 0.4148 | 0.7957 | 0.8372 | 0.7046 | 0.64 | 0.4409 | 1.3832 |
| 10.5 | 0.4167 | 0.8301 | 0.8561 | 0.73335 | 0.6549 | 0.44535 | 1.3952 |
| 11 | 0.4186 | 0.8645 | 0.875 | 0.7621 | 0.6698 | 0.4498 | 1.4072 |
| 11.5 | 0.4209 | 0.897 | 0.8868 | 0.79 | 0.6845 | 0.4525 | 1.4184 |
| 12 | 0.4224 | 0.927 | 0.896 | 0.8241 | 0.6987 | 0.4563 | 1.4293 |
| 12.5 | 0.4243 | 0.9517 | 0.9033 | 0.8541 | 0.7118 | 0.4599 | 1.4396 |
| 13 | 0.4262 | 0.9725 | 0.9107 | 0.8849 | 0.7253 | 0.4638 | 1.4496 |
| 13.5 | 0.4273 | 0.9899 | 0.9174 | 0.9111 | 0.7359 | 0.4667 | 1.4596 |
| 14 | 0.4287 | 1.0031 | 0.9197 | 0.9359 | 0.7482 | 0.4712 | 1.4681 |
| 14.5 | 0.431 | 1.0267 | 0.9229 | 0.9627 | 0.7574 | 0.4739 | 1.4773 |
| 15 | 0.4321 | 1.0406 | 0.9258 | 0.9836 | 0.7647 | 0.4778 | 1.4854 |
| 15.5 | 0.4334 | 1.05 | 0.9322 | 0.9972 | 0.7769 | 0.4806 | 1.4941 |
| 16 | 0.4346 | 1.06345 | 0.9357 | 1.0078 | 0.78515 | 0.48185 | 1.50215 |
| 16.5 | 0.4358 | 1.0769 | 0.9392 | 1.0184 | 0.7934 | 0.4831 | 1.5102 |
| 17 | 0.4363 | 1.0841 | 0.9467 | 1.0296 | 0.8007 | 0.486 | 1.5166 |
| 17.5 | 0.4379 | 1.1062 | 0.9549 | 1.0365 | 0.8111 | 0.4843 | 1.5233 |
| 18 | 0.4381 | 1.1201 | 0.96 | 1.047 | 0.8221 | 0.4924 | 1.5284 |
| 18.5 | 0.4398 | 1.1494 | 0.9675 | 1.0566 | 0.8325 | 0.4923 | 1.5346 |
| 19 | 0.4419 | 1.1729 | 0.9743 | 1.0608 | 0.8432 | 0.4949 | 1.54 |
| 19.5 | 0.4416 | 1.1942 | 0.9797 | 1.0669 | 0.8542 | 0.4959 | 1.5457 |
| 20 | 0.4434 | 1.2233 | 0.9857 | 1.0761 | 0.8671 | 0.496 | 1.5505 |
| 20.5 | 0.4412 | 1.2352 | 0.9911 | 1.086 | 0.8807 | 0.4974 | 1.556 |
| 21 | 0.445 | 1.27 | 0.9965 | 1.0943 | 0.8928 | 0.4963 | 1.5613 |
| 21.5 | 0.446 | 1.2882 | 1.0008 | 1.0985 | 0.9062 | 0.4947 | 1.5656 |
| 22 | 0.4439 | 1.2935 | 1.0031 | 1.1062 | 0.924 | 0.4999 | 1.571 |
| 22.5 | 0.4457 | 1.312 | 1.0088 | 1.1152 | 0.9363 | 0.5006 | 1.5763 |
| 23 | 0.449 | 1.3311 | 1.0121 | 1.1225 | 0.9534 | 0.5013 | 1.5807 |
| 23.5 | 0.4492 | 1.3389 | 1.0144 | 1.1255 | 0.9711 | 0.4999 | 1.5849 |
| 24 | 0.4485 | 1.3477 | 1.0206 | 1.143 | 0.9902 | 0.5035 | 1.5904 |

A further test of the growth of Bifidobacterium lactis BI-04 on a range of probiotics was carried out using the general method. The results for this further test are shown in Table 4 and the growth curve obtained from this data is shown in FIG. 2.

TABLE 4

| Time | No | | XOS | XOS | XOS | |
|---|---|---|---|---|---|---|
| (h) | carbohydrates | Glucose | dp2 | dp2-10 | Longlive | FOS |
| 0 | 0.3581 | 0.358 | 0.3913 | 0.5828 | 0.3642 | 0.3368 |
| 0.5 | 0.3627 | 0.3615 | 0.393 | 0.5903 | 0.3671 | 0.3414 |
| 1 | 0.3664 | 0.3671 | 0.3946 | 0.6235 | 0.3705 | 0.3502 |
| 1.5 | 0.3696 | 0.3772 | 0.397 | 0.6352 | 0.3754 | 0.3628 |
| 2 | 0.3733 | 0.3899 | 0.3997 | 0.6462 | 0.3843 | 0.3811 |
| 2.5 | 0.3785 | 0.407 | 0.4032 | 0.6598 | 0.395 | 0.4091 |
| 3 | 0.3864 | 0.4301 | 0.4096 | 0.6778 | 0.4117 | 0.4515 |
| 3.5 | 0.3952 | 0.4552 | 0.4182 | 0.7058 | 0.4365 | 0.5108 |
| 4 | 0.4005 | 0.4845 | 0.4328 | 0.7435 | 0.4738 | 0.5891 |
| 4.5 | 0.4044 | 0.5215 | 0.4523 | 0.792 | 0.5177 | 0.6713 |
| 5 | 0.40755 | 0.57115 | 0.4796 | 0.8563 | 0.5716 | 0.7465 |
| 5.5 | 0.4107 | 0.6208 | 0.5187 | 0.9315 | 0.6384 | 0.7957 |
| 6 | 0.4126 | 0.6769 | 0.569 | 1.0071 | 0.7188 | 0.8201 |
| 6.5 | 0.4162 | 0.7519 | 0.6301 | 1.0809 | 0.8044 | 0.8394 |
| 7 | 0.4196 | 0.824 | 0.7019 | 1.1513 | 0.8907 | 0.8563 |
| 7.5 | 0.4216 | 0.8944 | 0.7757 | 1.2103 | 0.9618 | 0.8705 |
| 8 | 0.4244 | 0.9606 | 0.8477 | 1.2579 | 1.0258 | 0.8905 |
| 8.5 | 0.426 | 1.0277 | 0.9089 | 1.2976 | 1.0767 | 0.9065 |
| 9 | 0.4287 | 1.0871 | 0.9692 | 1.325 | 1.1254 | 0.9258 |
| 9.5 | 0.4292 | 1.1209 | 1.0177 | 1.3472 | 1.1667 | 0.9404 |
| 10 | 0.4317 | 1.1556 | 1.0546 | 1.3657 | 1.2037 | 0.9553 |
| 10.5 | 0.43345 | 1.18865 | 1.0859 | 1.3798 | 1.2345 | 0.9683 |
| 11 | 0.4352 | 1.2217 | 1.112 | 1.3934 | 1.2589 | 0.9795 |
| 11.5 | 0.4393 | 1.2528 | 1.1356 | 1.4005 | 1.2748 | 0.9914 |
| 12 | 0.4383 | 1.2826 | 1.1536 | 1.4092 | 1.2863 | 1.0032 |
| 12.5 | 0.441 | 1.3101 | 1.1681 | 1.4132 | 1.2984 | 1.0195 |
| 13 | 0.4427 | 1.3368 | 1.1829 | 1.4195 | 1.3051 | 1.038 |
| 13.5 | 0.4448 | 1.3586 | 1.1931 | 1.4241 | 1.3102 | 1.0569 |
| 14 | 0.4463 | 1.3745 | 1.2047 | 1.4281 | 1.3171 | 1.0784 |
| 14.5 | 0.448 | 1.388 | 1.2137 | 1.4328 | 1.3191 | 1.0976 |
| 15 | 0.45 | 1.4068 | 1.2228 | 1.4362 | 1.3257 | 1.1176 |
| 15.5 | 0.4556 | 1.4166 | 1.231 | 1.4412 | 1.3294 | 1.1393 |
| 16 | 0.4564 | 1.43035 | 1.2394 | 1.4429 | 1.3348 | 1.1568 |
| 16.5 | 0.4572 | 1.4441 | 1.246 | 1.4479 | 1.3392 | 1.1715 |
| 17 | 0.4591 | 1.4486 | 1.2537 | 1.4534 | 1.3446 | 1.1827 |
| 17.5 | 0.4588 | 1.4526 | 1.2596 | 1.4545 | 1.3505 | 1.1938 |
| 18 | 0.4626 | 1.4654 | 1.2651 | 1.4578 | 1.354 | 1.2023 |
| 18.5 | 0.4657 | 1.4724 | 1.2705 | 1.46 | 1.3601 | 1.2087 |
| 19 | 0.4666 | 1.472 | 1.2748 | 1.464 | 1.3647 | 1.213 |
| 19.5 | 0.4682 | 1.4796 | 1.2791 | 1.4681 | 1.3688 | 1.2167 |
| 20 | 0.4714 | 1.4864 | 1.2829 | 1.4703 | 1.3727 | 1.2195 |
| 20.5 | 0.4715 | 1.491 | 1.2866 | 1.4703 | 1.3792 | 1.2211 |
| 21 | 0.4729 | 1.4946 | 1.2887 | 1.4734 | 1.3846 | 1.2262 |
| 21.5 | 0.4784 | 1.4988 | 1.2927 | 1.4754 | 1.3861 | 1.2253 |
| 22 | 0.4833 | 1.5015 | 1.2951 | 1.4767 | 1.3913 | 1.2315 |
| 22.5 | 0.4861 | 1.5046 | 1.2964 | 1.4785 | 1.3919 | 1.2305 |
| 23 | 0.491 | 1.5061 | 1.299 | 1.4823 | 1.3961 | 1.2327 |
| 23.5 | 0.4956 | 1.5092 | 1.3027 | 1.4826 | 1.4022 | 1.2338 |
| 24 | 0.5008 | 1.5092 | 1.3034 | 1.4827 | 1.4048 | 1.2353 |

Similar growth curves were obtained for other B. lactis strains.

Growth of Lactobacillus Acidophilus

Two experiments were carried out to determine the growth of Lactobacillus acidophilus on a range of probiotics was carried out using the general method. The results for experiment 1 are shown in FIG. 4, and for experiment 2 are shown in FIG. 5.

It can be seen from a comparison of these curves that xylo-oligosaccharides support the growth of B. lactis much better. In contrast, Fructo-oligosaccharide would support the growth of both. Hence xylo-oligosaccharides are more selective.

Bifidobacteria and Single Carbohydrates

The rate of bacterial growth for various strains of bifidobacteria on a range of single carbohydrates was determined in accordance with the general method. The average area under the growth curve without medium was as indicated in Table 5 and in FIG. 6.

TABLE 5

| | Glucose | FOS | Longlive | dp2 | dp2-10 |
|---|---|---|---|---|---|
| lactis 420 | 836 | 504 | 621 | 456 | 332 |
| lactis BI-04 | 629 | 530 | 485 | 310 | 199 |
| lactis Danone | 397 | 396 | 405 | 285 | 375 |
| lactis Howaru | 978 | 440 | 692 | 341 | 364 |
| lactis Bb-12 | 495 | 384 | 530 | 399 | 366 |
| lactis BI-07 | 933 | 817 | 386 | 287 | 100 |
| bifidum BB-02 | 512 | 313 | 41 | 0 | 0 |
| longum 913 | 443 | 6 | 27 | 0 | 0 |
| longum KC-1 | 553 | 251 | 0 | 0 | 0 |

A further experiment was carried out to determine the rate of bacterial growth for a wider variety of strains of bifidobacteria on a range of single carbohydrates in accordance with the general method. The average area under the growth curve without medium was as indicated in Table 6 and in FIG. 7.

TABLE 6

| | | XOS | XOS | XOS | |
|---|---|---|---|---|---|
| | Glucose | dp2 | dp2-10 | Longlive | FOS |
| B. breve Bb-03 | 561 | 6 | 7 | 62 | 397 |
| B. infantis DSM 20088 | 800 | 90 | 67 | 316 | 1623 |
| B. adolescentis | 1028 | 697 | 368 | 829 | 1123 |
| DSM 20083 | | | | | |
| B. longum DSM 20019 | 1143 | 33 | −124 | 89 | 810 |
| B. longum 913 | 1317 | 144 | 58 | 98 | 299 |
| B. longum KC-1 | 1194 | 2 | −38 | 95 | 489 |
| B. lactis BI-07 | 1072 | 663 | 320 | 832 | 815 |
| B. lactis 420 | 994 | 693 | 661 | 906 | 761 |
| B. lactis BI-04 | 951 | 715 | 818 | 893 | 761 |
| B. lactis Bb-12 | 838 | 617 | 544 | 874 | 770 |
| B. lactis DN173010 | 772 | 612 | 595 | 544 | 473 |
| B. lactis HN019 | 1062 | 801 | 856 | 818 | 779 |

All B. lactis utilized the xylo-oligosaccharides Longlive, dp2, and dp2-10 to a large extent. Most of the tested bifidobacteria were able to grow on FOS to similar levels, or even higher, than on glucose, thus XOS is more selective than FOS.

Lactobacilli and S. thermophilus with Single Carbohydrates

Two experiments were carried out to measure the rate of bacterial growth for various strains of Lactobacilli and for S. thermophilus with a range of single carbohydrates. The rate of bacterial growth was determined in accordance with the general method. For the first experiment, the average area under the growth curve without medium was as indicated in Table 7 and in FIG. 8.

TABLE 7

| | Glucose | FOS | Longlive | dp2 | dp2-10 |
|---|---|---|---|---|---|
| L. acidophilus | 1305 | 1135 | 215 | 136 | 207 |
| L. bulgaricus | 716 | 0 | 0 | 0 | 157 |
| L. paracasei | 761 | 685 | 0 | 0 | 0 |
| L. rhamnosus | 948 | 41 | 66 | 0 | 0 |
| S. thermophilus | 425 | 93 | 4 | 3 | 161 |

For the second experiment, the average area under the growth curve without medium was as indicated in Table 8 and in FIG. 9.

TABLE 8

| | | | XOS | XOS | |
|---|---|---|---|---|---|
| | Glucose | XOS dp2 | dp2-10 | Longlive | FOS |
| L. acidophilus | 1099 | 136 | −46 | 215 | 1135 |
| NCFM 145 | | | | | |
| L. bulgaricus | 909 | −43 | 280 | −42 | −20 |
| L. paracasei Lpc-37 | 877 | −91 | −191 | −103 | 685 |
| L. rhamnosus | 1041 | −57 | −121 | 66 | 41 |
| HN001 | | | | | |
| Strep. thermophilus | 1066 | 3 | 51 | 4 | 93 |

Whilst all these bacteria fermented with glucose, they did not ferment to any great extent with xylo-oligosaccharides. FOS enhanced the growth of Lactobacillus acidophilus NCFM and paracasei Lpc-37 to a large extent.

Pathogens and Single Carbohydrates

Two experiments were carried out to measure the growth rate for various pathogens and other microbes of colonic origin with a range of single carbohydrates. The growth rate was determined in accordance with the general method. For the first experiment, the average area under the growth curve without medium was as indicated in Table 9 and in FIG. 10.

TABLE 9

| | Glucose | FOS | Longlive | dp2 | dp2-10 |
|---|---|---|---|---|---|
| C. perfringens | 1006 | 120 | 0 | 0 | 0 |
| E. coli O1:57 | 701 | 130 | 44 | 179 | 0 |
| E. limosum | 131 | 6 | 0 | 0 | 0 |
| S. typhimurium | 819 | 108 | 186 | 181 | 0 |
| S. aureus | 500 | 336 | 0 | 0 | 0 |
| S. epidermis | 497 | 246 | 0 | 32 | 0 |

For the second experiment, the average area under the growth curve without medium was as indicated in Table 10 and in FIG. 11.

TABLE 10

| | | XOS | | Xos | |
|---|---|---|---|---|---|
| | Glucose | dp2 | XOS dp2-10 | Longlive | FOS |
| Eub. limosum | 677 | 75 | 232 | 51 | 626 |
| Eub. biforme | 235 | 19 | 101 | 19 | 59 |
| Bact. vulgatus | 410 | 63 | 75 | 99 | 592 |
| Bact. fragilis | 226 | 120 | 116 | 81 | 228 |
| Cl. difficile | 346 | 31 | 83 | 115 | 69 |
| Cl. perfringens | 1059 | −78 | −142 | −38 | 120 |
| E. coli | 769 | 114 | −54 | 29 | 162 |
| Salm. typhimurium | 744 | 23 | −8 | 74 | 3 |
| Staph. epidermis | 594 | 32 | −39 | −4 | 246 |
| Staph. aureus | 247 | −44 | −186 | −111 | 336 |

Xylo-oligosaccharides exhibited a lower growth rate for these pathogens than did FOS or glucose. FOS enhanced the growth of Eubacterium limosum, Bacteroides vulgatus and Bact. fragilis, and Staphylococcus aureus to the same levels as glucose, the positive control. The growth of potentially pathogenic microbes, Escherichia coli, Clostridium perfringens, Salmonella typhimurium and Staphylococcus aureus and Staph. epidermis was even inhibited in comparison to the growth on carbohydrate free MRS or TSB media.

Colon Simulations

A number of colon simulations were carried out using a semi-continuous four-channel colon simulator model consisting of four parallel units V1 to V4. The conditions of the units were adjusted to represent different compartments of the human colon; from V1 representing the cecum/ascending colon, to V4 representing the distal colon/rectum. The colon simulations were carried out as described by Mäkivuokko, H., et al. Nutr. Cancer, 52, (2005), 94-104; and Mäkivuokko, H., et al. Biosci. Biotechnol. Biochem., 70, (2006), 2056-2063.

General Method

The simulator unit was kept anaerobic from the medium vessel feeding the first vessel (V1) to the last vessel (V4) by gassing the vessels with anoxic N₂. Each four-stage unit had 1 g of the appropriate carbohydrate dissolved to 50 ml in the sterile simulator medium, (see Macfarlane, G. T., et al. Microb. Ecol., 35, (1998), 180-187), and sealed in a glass serum bottle inside the anaerobic cabinet. For the control simulation units, 50 ml of the sterile simulator medium was similarly sealed in a glass serum bottle. All the vessels of the simulator units were inoculated anaerobically with samples of the relevant bacteria.

Colon Simulation—Bifidobacterium

The mean results (plus or minus standard error of mean, ±SE) for a colon simulation for Bifidobacteria with a range of single carbohydrates are given in FIG. 12.

Colon Simulation—B. lactis

The results for a colon simulation for Bifidobacterium lactis with a range of single carbohydrates are given in FIG. 13.

Colon Simulation—B. lonqum

The results for a colon simulation for Bifidobacterium longum with a range of single carbohydrates are given in FIG. 14.

Colon Simulation—C. perfringens

The results for a colon simulation for Clostridium perfringens with a range of single carbohydrates are given in FIG. 15.

Colon Simulation—Short Chain Fatty Acids

The results for a colon simulation for total short chain fatty acids with a range of single carbohydrates are given in FIG. 16. The term total short chain fatty acids refers to C1 to C5 chain fatty acids, in particular, to acetic, propionic and butyric acid.

### CONCLUSION

It can be seen from these colon simulations that the xylo-oligosaccharide species show much greater selectivity for Bifidobacterium lactis than for other species, such as B. longum and Cl. perfringens.

### Example 1

Yoghurt containing 10⁶-10⁸ CFU/ml of B. lactis HN0019 and 5 g per serving of xylo-oligosaccharides (dp2-10). The xylo-oligosaccharides were added after a standard fermentation process.

This Example may be varied to use any B. lactis strain or any xylo-oligosaccharide sample as described herein. In addition, the amount of xylo-oligosaccharides may be varied to use from 2 to 10 g per serving.

### Example 2

Nutrition bar containing 10⁷-10¹⁰ CFU of B. lactis HN0019 and 5 g per serving of xylo-oligosaccharides (dp2-10).

This Example may be varied to use any B. lactis strain or any xylo-oligosaccharide sample as described herein. In addition, the amount of xylo-oligosaccharides may be varied to use from 2 to 10 g per serving.

### Example 3

A powdered beverage containing 10⁷-10¹⁰ CFU of B. lactis HN0019 strain per serving and 5 g per serving of xylo-oligosaccharides (dp2-10). Where water activity is maintained below 0.5.

This Example may be varied to use any B. lactis strain or any xylo-oligosaccharide sample as described herein. In addition, the amount of xylo-oligosaccharides may be varied to use from 2 to 10 g per serving.

### Example 4

An infant formula containing 10⁷-10¹⁰ CFU of B. lactis HN0019 strain per serving and 5 g per serving of xylo-oligosaccharides (dp2-10). Where water activity is maintained below 0.5.

This Example may be varied to use any B. lactis strain or any xylo-oligosaccharide sample as described herein. In addition, the amount of xylo-oligosaccharides may be varied to use from 2 to 10 g per serving.

### Example 5

A milk powder containing 10⁷-10¹⁰ CFU of B. lactis HN0019 strain per serving and 5 g per serving of xylo-oligosaccharides (dp2-10). The xylo-oligosaccharides being added as a solution before spray drying.

This Example may be varied to use any B. lactis strain or any xylo-oligosaccharide sample as described herein. In addition, the amount of xylo-oligosaccharides may be varied to use from 2 to 10 g per serving.

### Example 6

A milk powder containing 10⁷-10¹⁰ CFU of B. lactis HN0019 strain per serving and 5 g per serving of xylo-oligosaccharides (dp2-10). The xylo-oligosaccharides being added after spray drying.

This Example may be varied to use any B. lactis strain or any xylo-oligosaccharide sample as described herein. In addition, the amount of xylo-oligosaccharides may be varied to use from 2 to 10 g per serving.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims

1. A composition comprising:
(a) one or more live Bifidobacterium lactis strains; and
(b) a saccharide component;
wherein the saccharide component comprises xylo-oligosaccharides with a degree of polymerisation of from 2 to 100.

2. A composition according to claim 1, wherein the proportion of xylo-oligosaccharides with a polymerisation degree of 2 in saccharide component (b) is at least 50%.

3. A composition according to claim 1, wherein the proportion of xylo-oligosaccharides with a polymerisation degree of 2 in saccharide component (b) is at least 70%.

4. A composition according to claim 1, wherein the proportion of xylo-oligosaccharides with a polymerisation degree of 3 in saccharide component (b) is at least 10%.

5. A composition according to claim 1, wherein the proportion of xylo-oligosaccharides with a polymerisation degree of at least 4 in saccharide component (b) is at least 30%.

6. A composition according to claim 1, wherein the xylo-oligosaccharides have a degree of polymerisation of from 2 to 10.

7. A composition according to claim 1, wherein the proportion of xylo-oligosaccharides with a polymerisation degree of at least 5 in saccharide component (b) is greater than 50%.

8. A composition according to claim 1, wherein the xylo-oligosaccharides is xylan.

9. A composition according to claim 1, wherein the one or more live Bifidobacterium lactis strains are selected from B. lactis BI-04, B. lactis Bi-07, B. lactis 420, B. lactis DN 173 010, B. lactis HN019, B. lactis Bb-12, B. lactis DR10, B. lactis DSM10140, B. lactis LKM512, B. lactis DSM 20451 and mixtures thereof.

10. A composition according to claim 1, wherein the composition is a food composition.

11. A composition according to claim 10, wherein the food composition comprises from 1×10⁶ to 1×10¹² Colony Forming Units per serving of Bifidobacterium lactis strains.

12. A product for oral consumption comprising a composition according to claim 1 wherein the product is selected from dry oral supplements, liquid oral supplements, milk, yoghurt, cheese, ice-creams, products made of cereals, infant formulae, powdered beverages, confectionary, nutrition bars and milk powders.

13. A kit comprising,
a first vessel comprising one or more live Bifidobacterium lactis strains; and
a second vessel comprising a saccharide component wherein the saccharide component comprises xylo-oligosaccharides with a degree of polymerisation of from 2 to 100.

14. A kit according to claim 13 wherein the Bifidobacterium lactis strains are incorporated into a pill or into yoghurt.

15. A kit according to claim 13 wherein the xylo-oligosaccharides are incorporated into a foodstuff selected from fruit juice and products made of cereals.

16. Use of a composition according to claim 1 in the manufacture of a medicament to selectively increase the colonisation and/or the activity of Bifidobacterium lactis in the intestine of a subject.

17. Use of a composition according to claim 1, in the manufacture of a medicament to reduce or inhibit the colonisation of Clostridium perfingens in the intestine of a subject.

18. Use of a composition according to claim 1, in the manufacture of a medicament to increase the levels of Bifidobacterium lactis in a fermented food.

19. Use of a composition according to claim 1, in the manufacture of a medicament to reduce atopic eczema.

20. Use of a composition according to claim 1, in the manufacture of a medicament for the treatment of diarrhea.

21. Use of a composition according to claim 1, in the manufacture of a medicament to enhance immune function.

22. Use of a composition according to claim 1, in the manufacture of a medicament to improve bowel function.
